(19) United States
(12) Patent Application Publication
(10) Pub. No.: US 2024/0000672 A1
(43) Pub. Date: Jan. 4, 2024

(54) SOLID COMPOSITION COMPRISING A COMBINATION OF PARTICULAR ANIONIC SURFACTANTS AND AT LEAST ONE POLYMERIC ORGANIC FILLER

(71) Applicant: L'OREAL, Paris (FR)

(72) Inventors: Christelle MORVAN, Saint Ouen (FR); Andreia TEIXEIRA, Saint Ouen (FR)

(21) Appl. No.: 18/255,656

(22) PCT Filed: Dec. 3, 2021
(86) PCT No.: PCT/EP2021/084249

§ 371 Date: Jun. 2, 2023

(30) Foreign Application Priority Data
Dec. 3, 2020 (FR) 2012597

(51) Int. Cl. A61K 8/02; A61K 8/46; A61K 8/44; A61K 8/73; A61Q 5/02; A61Q 5/12

(52) U.S. Cl. CPC A61K 8/022; A61K 8/466; A61K 8/442; A61K 8/732; A61K 2800/87; A61Q 5/02; A61Q 5/12; A61K 2800/596; A61K 2800/5426; A61K 8/737

(57) ABSTRACT

The present invention relates to a solid composition intended in particular for washing and/or conditioning keratin fibres, notably human keratin fibres such as the hair, and which comprises a particular combination of at least two anionic surfactants, of which one is of sulfonate type and the other is of carboxylate type, in the presence of at least one particular filler. The invention also relates to a packaging article comprising said solid composition, and also to cosmetic processes for treating keratin fibres, in particular human keratin fibres such as the hair, using said solid composition or said packaging article. The invention also relates to the use of said solid composition or of said packaging article for washing and/or conditioning keratin fibres, in particular human keratin fibres such as the hair.

The present invention relates to a solid composition intended in particular for washing and/or conditioning keratin fibres, notably human keratin fibres such as the hair, and which comprises a particular combination of at least two anionic surfactants, of which one is of sulfonate type and the other is of carboxylate type, in the presence of at least one particular filler.

The invention also relates to a packaging article comprising said solid composition, and also to cosmetic processes for treating keratin fibres, in particular human keratin fibres such as the hair, using said solid composition or said packaging article.

The invention also relates to the use of said solid composition or of said packaging article for washing and/or conditioning keratin fibres, in particular human keratin fibres such as the hair.

In the field of hair hygiene, products for washing keratin fibres are generally intended to cleanse said fibres while at the same time giving them good cosmetic properties. Conventional products, such as shampoos, are usually in more or less thickened liquid form. However, on account of their liquid texture, these products may have various drawbacks, and may notably prove to be difficult to measure out.

The reason for this is that the more liquid they are, the greater their tendency to escape between the fingers, making them difficult to measure out and leading to waste. These products may also escape from their packaging, which is a source of inconvenience to the consumer when these products come into contact with clothing or objects, for example when travelling.

In order to modify the texture of these products, and notably to make it more compact, thickeners are generally used. However, the addition of these compounds usually comes at the expense of the cosmetic effects of the compositions. The use of these thicker compositions moreover necessitates a large amount of rinsing water in order to remove the surplus of product on the fibres. Now, in many countries where access to water is restricted, the rinsing time and consequently the amount of water required to properly rinse off the product are key indicators of the working qualities of a composition.

In order to overcome some of these problems, novel solid cosmetic formulations, notably shampoos in the form of solid granules or powder, have been developed. However, these novel formulations are not always entirely satisfactory. Those which are in loose powder form may, indeed, pose problems of volatility, uptake and/or measuring out, whereas those which are in the form of agglomerates, for instance granules, may have a tendency to disintegrate or break down with difficulty in the presence of water. Thus the latter do not always make it possible to obtain a rapid start of foaming and/or a satisfactory abundance of foam, having a negative impact on their use and their spreading on keratin fibres. They may also be difficult to remove on rinsing and may occasionally even leave residues on the fibres which the consumer finds unpleasant. These formulations may also not be entirely satisfactory in terms of cosmetic performance qualities, notably in terms of suppleness, feel, softness, sheen and disentangling.

Thus, there is a real need to provide a composition in solid form which has an improved environmental profile, i.e. which requires little water throughout its use. The composition must not only be easy to take up, break down easily and have good foaming properties, notably in terms of the start of foaming and the foam abundance and density, but must also rinse out quickly without leaving residues on the keratin fibres.

The composition must also have good detergent power while at the same time affording satisfactory cosmetic properties, notably in terms of suppleness, feel, softness, sheen and disentangling.

It has now been found that a solid composition comprising a particular combination of at least two anionic surfactants, of which one is of sulfonate type and the other is of carboxylate type, in a particular weight ratio, in the presence of an amphoteric or zwitterionic surfactant and a polymeric organic filler makes it possible to achieve the objectives presented above, and notably to propose a composition in solid form which combines good detergent power with improved foam properties, without, however, requiring large amounts of water.

One subject of the present invention is a solid composition comprising:
(i) one or more anionic surfactants of sulfonate type,
(ii) one or more anionic surfactants of carboxylate type,
(iii) one or more amphoteric or zwitterionic surfactants,
(iv) one or more polymeric organic fillers, where the weight ratio between the total content of anionic surfactant(s) of carboxylate type (ii) and the total content of anionic surfactant(s) of sulfonate type (i) is greater than or equal to 0.6,
   the composition comprising a water content of less than 5% by weight, relative to the total weight of the composition.

The particular combination of the compounds of the invention makes it possible to obtain a solid composition that is easy to take up, to handle and to measure out. Specifically, the composition thus obtained has a cohesion or granulation such that the uptake and measuring-out properties are improved. The composition can then be packaged in single-dose form, which is a form that is particularly advantageous, for example, when travelling or performing a sporting activity (lightened bags, limited risks of leakage, reduced waste).

This composition also breaks down rapidly on contact with water and readily and quickly produces a firm, creamy and abundant foam, the quality of which is comparable to that of the foam obtained with a conventional liquid shampoo composition. This foam can then be easily and uniformly distributed on the keratin fibres.

Moreover, the composition of the invention rinses out rapidly without leaving unpleasant residues on the fibres and gives them a natural, clean feel after rinsing. Fibres treated with the composition of the invention also have good cosmetic properties, notably in terms of softness, suppleness and feel. They also have good strand separation and are thus easier to disentangle.

A subject of the present invention is also a cosmetic treatment process, notably for washing and/or conditioning keratin fibres, in particular human keratin fibres such as the hair, comprising the application to said keratin fibres of a solid composition as defined previously, the solid composition being applied directly to said keratin fibres or after having been moistened beforehand with water.

The present invention also relates to the use of a solid composition as defined previously for washing and/or conditioning keratin fibres, in particular human keratin fibres such as the hair.

The present invention also relates to a packaging article comprising:
an envelope defining at least one cavity, the envelope comprising one or more water-soluble and/or liposoluble compounds;
a solid composition as defined above;
it being understood that the solid composition is in one of the cavities defined by the envelope.

This packaging article notably solves the problems of measuring out of the solid composition. It also facilitates its storage and transportation. In particular, the packaging article of the invention affords better protection of the composition against moisture.

The packaging article may also make it possible to obtain a final keratin fibre washing and/or conditioning composition that is more thickened in the hand, which may be in cream form. It may also act as a foam booster. Specifically, the volume of foam obtained after dilution of the packaging article may be greater than the volume of foam obtained after dissolution of the solid composition alone.

The present invention also relates to the use of a packaging article as defined previously for washing and/or conditioning keratin fibres, in particular human keratin fibres such as the hair.

A subject of the present invention is also a cosmetic treatment process, notably for washing and/or conditioning keratin fibres, in particular human keratin fibres such as the hair, comprising a step of using a packaging article as defined above.

Preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to stand,
iv) rinsing said keratin fibres, and
v) optionally drying said keratin fibres.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

The solid composition according to the present invention comprises a water content of less than 5% by weight, preferably less than 4% by weight, more preferably less than 3% by weight, relative to the total weight of the composition. Even more preferably, the solid composition according to the invention comprises a water content of 0% by weight, relative to the total weight of the composition. Such a composition will be referred to as an “anhydrous composition” in the following description. In other words, the solid composition according to the present invention corresponds to an anhydrous solid composition in the following description.

In particular, the composition does not comprise any water added during its preparation, the residual water that may be present possibly originating from the starting materials used during the preparation.

The anhydrous solid composition according to the invention may be in powder, paste, particle (for example spherical particles such as small beads or granules), compressed tablet, stick or cake form. Preferably, the composition according to the invention is in the form of a powder or of particles.

The term “powder” means a composition in pulverulent form, which is preferably essentially free of dust (or fine particles). In other words, the particle size distribution of the particles is such that the weight content of particles which have a size of less than or equal to 50 micrometres (content of fines), preferably less than or equal to 45 micrometres (content of fines) is advantageously less than or equal to 5% by weight, preferably less than 3% by weight and more particularly less than 1% by weight, relative to the total weight of particles (particle size evaluated using a Retsch AS 200 Digit particle size analyser; oscillation height: 1.25 mm/screening time: 5 minutes).

The term “paste” means a composition having a viscosity of greater than 5 poises (0.5 Pa·s), and preferably greater than 10 poises (1 Pa·s), measured at 25° C. and at a shear rate of 1 s⁻¹; this viscosity possibly being determined using a cone-plate rheometer.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred.

Advantageously, the size of the powders or particles is, in its largest dimension, between 45 μm and 5 mm, preferably between 50 μm and 2 mm, more preferentially between 50 μm and 1 mm, and better still between 60 μm and 600 μm.

When the anhydrous solid composition according to the invention is not in powder or particle form, it preferably has a penetration force at 25° C. and 1 atm of greater than or equal to 200 g, preferably greater than or equal to 300 g, more preferentially greater than or equal to 400 g and better still greater than or equal to 500 g. The penetration force is determined by penetrometry. The texture analysis measurements are performed at 25° C. using a Stable Micro Systems TA.XT Plus texturometer. The penetrometry experiments are performed with a metal rod equipped with a screwed end piece, said end piece being a P/2N needle of 2 mm for the top part, connected to the measuring head. The piston penetrates into the sample at a constant speed of 1 mm/s, to a depth of 5 mm. The force exerted on the piston is recorded and the mean value of the force is calculated.

The anhydrous solid composition according to the invention may be in the form of a compressed anhydrous solid composition, notably compressed using a manual or mechanical press. Preferably, the hardness of the compressed anhydrous solid composition is between 10 and 300 N, more preferentially between 15 and 200 N and better still between 15 and 100 N.

The density of the anhydrous solid composition according to the present invention is preferably between 0.1 and 1, more preferentially between 0.2 and 0.8 and better still between 0.3 and 0.6.

A given amount (mass, m) of powder is placed in a measuring cylinder. The measuring cylinder is then automatically tapped 2500 times. The volume (v) thus obtained is read on the measuring cylinder and the density (d) is then determined according to the formula d=m/v.

The Anionic Surfactant(s) of Sulfonate Type

The anhydrous solid composition according to the present invention comprises one or more anionic surfactants of sulfonate type.

In the sense of the present invention, the term “anionic surfactant of sulfonate type” means an anionic surfactant containing one or more sulfonic or sulfonate functions (—SO₃H or —SO₃⁻), which may optionally contain one or more carboxylic or carboxylate functions (—COOH or —COO⁻) and does not contain any sulfate functions.

Surfactants of this kind may advantageously be chosen from alkylsulfonates, alkylamidesulfonates, alkylarylsulfonates, alpha-olefinsulfonates, paraffinsulfonates, alkylsulfosuccinates, alkylethersulfosuccinates, alkylamidesulfosuccinates, alkylsulfoacetates, sulfolaurates, N-acyltaurates, acylisethionates, and salts thereof and mixtures thereof; the alkyl groups of these compounds contain notably from 8 to 30 carbon atoms, preferably from 8 to 26, and more preferentially from 10 to 22 carbon atoms; the aryl group denotes preferably a phenyl or benzyl group; these compounds may be polyoxyalkylenated, notably polyoxyethylenated and in that case contain preferably from 1 to 50 ethylene oxide units, and more preferentially from 2 to 10 ethylene oxide units.

Preferably, the anionic surfactant(s) of sulfonate type are chosen from N-acyltaurates, and notably N-acyl N-methyltaurates, acylisethionates, and sulfolaurates such as disodium 2-sulfolaurate, and also salts thereof and mixtures thereof.

The anionic surfactant(s) of sulfonate type may more preferentially be chosen advantageously from the compounds of formula (I):

R₁—COX—R₂—SO₃M (I)

formula (I), in which:
R₁ represents a linear or branched, preferably linear, alkyl group comprising from 8 to 30 carbon atoms, preferably from 8 to 26 carbon atoms, and more preferentially from 10 to 22 carbon atoms,
X represents an oxygen atom or a —N(CH₃)— or —NH— group, preferably an oxygen atom,
R₂ represents a linear or branched alkyl group comprising from 1 to 4 carbon atoms, and
M denotes a hydrogen atom, an ammonium ion, an ion obtained from an alkali metal or alkaline-earth metal, or an ion obtained from an organic amine.

The anionic surfactant(s) of sulfonate type, and notably those of formula (I) as defined above, may be used in salified or unsalified form.

Salts which may be used in particular are alkali metal salts, such as the sodium or potassium salts, ammonium salts, amine salts, amino alcohol salts or alkaline-earth metal salts, for example magnesium salts.

Amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferably, the anionic surfactant(s) of sulfonate type are chosen from acylisethionates and mixtures thereof, and more preferentially from acyl(C₈-C₃₀)isethionates and mixtures thereof, which are used in the form of salts, and even better still in the form of alkali metal or alkaline-earth metal salts, and more particularly of sodium or magnesium salts.

Examples of particularly preferred acyl(C₈-C₃₀) isethionate include notably the cocoylisethionates and the lauroyl methyl isethionates, more particularly in the form of sodium salts.

The total content of the anionic surfactant(s) of sulfonate type present in the anhydrous solid composition according to the invention ranges preferably from 1% to 30% by weight, more preferentially from 3% to 25% by weight, better still from 5% to 20% by weight, and even better still from 8% to 16% by weight, relative to the total weight of the composition.

In a preferred variant of the invention, the anionic surfactant(s) of sulfonate type are chosen from acyl(C₈-C₃₀) isethionates and mixtures thereof, and the total content of the acyl(C₈-C₃₀) isethionate(s) present in the anhydrous solid composition according to the invention ranges preferably from 1% to 30% by weight, more preferentially from 3% to 25% by weight, better still from 5% to 20% by weight, and even better still from 8% to 16% by weight, relative to the total weight of the composition.

The Anionic Surfactants of Carboxylate Type

The anhydrous solid composition according to the present invention further comprises one or more anionic surfactants of carboxylate type.

In the sense of the present invention, the term “anionic surfactant of carboxylate type” means an anionic surfactant containing one or more carboxylic or carboxylate functions (—COOH or —COO—), which does not contain any sulfonic or sulfonate function (—SO₃H or —SO₃) and does not contain any sulfate function.

Surfactants of these kinds may advantageously be chosen from acyllactates, N-acylglycinates, N-acylsarcosinates and N-acylglutamates, alkyl ether carboxylates, alkyl glucose carboxylates, alkyl glucoside tartrates and alkyl glucoside citrates, where the acyl or alkyl groups contain preferably from 8 to 30 carbon atoms, better still from 10 to 22 carbon atoms; and mixtures thereof; and also the unsalified forms of these compounds.

The anionic surfactant(s) of carboxylate type may preferably be chosen advantageously from the compounds of formula (II):

R—(OCH₂CH₂)ₙW—(CHY₁)ₚ—COOX (II)

formula (II), in which:
Y₁ denotes a hydrogen atom, a group (CH₂)_{q}COOX or a hydroxyl group;
W denotes an oxygen atom, a group (O-Glu-O)ᵣ—(COCH(Y₂)—(C(OH)COOX)ₜ)ₛ or a group CO—NR₃;
Y₂ denotes a hydrogen atom or a hydroxyl group;
R₃ denotes a hydrogen atom or a methyl group;
X denotes a hydrogen atom, an ammonium ion, an ion obtained from an alkali metal or alkaline-earth metal, or an ion obtained from an organic amine;
R denotes a linear or branched, preferably linear, alkyl group comprising from 8 to 30 carbon atoms, preferably from 8 to 26 carbon atoms, and more preferentially from 10 to 22 carbon atoms;
Glu denotes a divalent radical obtained from glucopyranose with removal of 2 hydroxyl groups;
p is equal to 0 or 1;
q denotes an integer ranging from 1 to 10;
n denotes an integer ranging from 0 to 50;
r denotes a number ranging from 1 to 10;
s is equal to 0 or 1; and
t is equal to 0 or 1.

The anionic surfactant(s) of carboxylate type (ii) are preferably chosen from the compounds of formula (II) for which:
Y₁ denotes a hydrogen atom or a group (CH₂)_{q}COOX;
W denotes a group CO—NR₁;
R₃ denotes a hydrogen atom or a methyl group;
X denotes a hydrogen atom, an ammonium ion, an ion obtained from an alkali metal or alkaline-earth metal, or an ion obtained from an organic amine;
R denotes a linear or branched, preferably linear, alkyl group comprising from 8 to 30 carbon atoms, preferably from 8 to 26 carbon atoms, and more preferentially from 10 to 22 carbon atoms;
p is equal to 0 or 1, preferably 0;
q denotes an integer ranging from 1 to 10;
n denotes an integer ranging from 0 to 50.

The anionic surfactant(s) of carboxylate type are more preferentially chosen from the compounds of formula (II) for which:
n=0, p=1, Y₁=H, W=CONH (N-acylglycinates),
n=0, p=1, W=CON(CH₃) and Y₁=H (N-acylsarcosinates), and
n=0, p=1, W=CONH and Y₁=CH₂CH₂COOX (N-acylglutamates).

The anionic surfactant(s) of carboxylate type, and notably those of formula (II) as defined above, may be employed in salified or unsalified form.

As salt it is possible more particularly to use alkali metal salts such as the sodium or potassium salts, ammonium salts, amine salts, amino alcohol salts or alkaline-earth metal salts, for example magnesium salts.

Amino alcohol salts include mono-, di- and triethanolamine salts, mono-, di- or triisopropanolamine salts, 2-amino-2-methyl-1-propanol salts, 2-amino-2-methyl-1,3-propanediol salts and tris(hydroxymethyl)aminomethane salts.

Preference is given to using the alkali metal or alkaline-earth metal salts, and more particularly the sodium or magnesium salts.

The anionic surfactants of carboxylate type are preferentially chosen from N-acyl(C₈-C₃₀) glutamates, and more particularly stearoylglutamates, lauroylglutamates and cocoylglutamates; N-acyl(C₈-C₃₀) sarcosinates, and more particularly palmitoylsarcosinates, stearoylsarcosinates, lauroylsarcosinates and cocoyl-sarcosinates; and mixtures thereof; more particularly in the form of alkali metal or alkaline-earth metal salts, ammonium salts, amine salts or amino alcohol salts.

With particular preference the anionic surfactant(s) of carboxylate type are chosen from N-acyl(C₈-C₃₀) glutamates, more particularly in the form of alkali metal or alkaline-earth metal salts, ammonium salts, amine salts or amino alcohol salts, and mixtures thereof.

The total content of the anionic surfactant(s) of carboxylate type present in the anhydrous solid composition according to the invention ranges preferably from 1% to 40% by weight, more preferentially from 2% to 35% by weight, better still from 5% to 30% by weight, and even better still from 10% to 25% by weight, relative to the total weight of the composition.

In a preferred variant of the invention, the anionic surfactant(s) of carboxylate type are chosen from N-acyl(C₈-C₃₀) glutamates and mixtures thereof, and the total content of the N-acyl(C₈-C₃₀) glutamate(s) present in the anhydrous solid composition according to the invention ranges preferably from 1% to 40% by weight, more preferentially from 2% to 35% by weight, better still from 5% to 30% by weight, and even better still from 10% to 25% by weight, relative to the total weight of the composition.

The weight ratio (R) between the total content of surfactant(s) of carboxylate type (ii) and the total content of surfactant(s) of sulfonate type (i) present in the anhydrous solid composition of the invention is greater than or equal to 0.6. Advantageously this weight ratio (R) is greater than or equal to 0.7, preferably greater than or equal to 0.8, more preferentially greater than or equal to 1.0, or even strictly greater than 1.0, and even better still greater than or equal to 1.1. Preferably this weight ratio (R) ranges from 0.6 to 5, more preferentially from 0.7 to 4.5, even better still from 0.8 to 4.0, better still even from 1.0 to 3.5, and more preferentially still from 1.1 to 3.0.

In a preferred embodiment, this weight ratio (R) is advantageously greater than or equal to 1, or even strictly greater than 1, this weight ratio (R) ranging preferably from 1 to 5, more preferentially from 1.5 to 3.5, and even better still from 2 to 3.

The anhydrous solid composition according to the invention is preferably free of anionic surfactant of sulfate type.

In the sense of the present invention, the term “anionic surfactant of sulfate type” means surfactants containing at least one group which is anionic or can be ionized to an anionic group, chosen from sulfate functions (—OSO₃H or —OSO₃).

The following anionic surfactants are therefore preferably not present in the anhydrous solid composition according to the invention: alkylsulfate salts, alkylamidosulfate salts, alkylethersulfate salts, alkylamidoethersulfate salts, alkylarylethersulfate salts, and monoglyceride-sulfate salts.

In the sense of the present invention, the term “free of” refers to a composition which does not contain (0%) these anionic surfactants of sulfate type or which contains less than 0.1% by weight of such surfactants, relative to the total weight of the composition.

The total content of the anionic surfactant(s), in other words notably the total content of anionic surfactants of sulfonate type (i) and of anionic surfactants of carboxylate type (ii), present in the anhydrous solid composition according to the invention is advantageously greater than or equal to 15% by weight, this content ranging preferably from 15% to 45% by weight, more preferentially from 20% to 40% by weight, and even better still from 25% to 35% by weight, relative to the total weight of the composition.

The Amphoteric or Zwitterionic Surfactants

The anhydrous solid composition according to the present invention also comprises one or more amphoteric or zwitterionic surfactants.

In particular, the amphoteric or zwitterionic surfactant(s), which are preferably non-silicone, used in the anhydrous solid composition according to the present invention may notably be derivatives of optionally quaternized secondary or tertiary aliphatic amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido (C₁-C₆)alkylbetaines and (C₈-C₂₀)alkylamido (C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

Rₐ—CONHCH₂CH₂—N⁺(R_{b})(R_{c})—CH₂COO⁻,M⁺,X⁻ (III)

in which formula (III):
Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
R_{b} represents a R-hydroxyethyl group;
Rₑ represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄))alkylarylsulfonates, in particular methyl sulfate and ethyl sulfate; or alternatively M⁺ and X⁻ are absent;

R_{a′}—CONHCH₂CH₂—N(B)(B′) (IV)

in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH) SO₃H or the group CH₂CH(OH) SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid R_{a′}—COOH which is preferably present in coconut kernel oil or in hydrolysed linseed oil, preferably R_{a′} an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (V)

R_{a″}—NHCH(Y″)—(CH₂)ₙCONH(CH₂)_{n′}—N(R_{d})(Rₑ) (V)

in which formula (V):
Y″ represents the group —COOH, —COOZ″ or —CH₂—CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in coconut kernel oil or in hydrolysed linseed oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine (C₈-C₂₀)alkylamido (C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido (C₃-C₈)alkylbetaines and mixtures thereof.

Preferentially, the amphoteric or zwitterionic surfactant(s) are chosen from (C₈-C₂₀)alkylbetaines, (C₈-C₂₀)alkylamido (C₃-C₈)alkylbetaines and mixtures thereof, and better still from (C₈-C₂₀)alkylamido (C₃-C₈)alkylbetaines and mixtures thereof.

The total content of the amphoteric or zwitterionic surfactant(s) present in the anhydrous solid composition according to the invention preferably ranges from 1% to 30% by weight, more preferentially from 2% to 25% by weight, and better still from 5% to 20% by weight, relative to the total weight of the composition.

The Polymeric Organic Fillers

The anhydrous solid composition according to the present invention also comprises one or more polymeric organic fillers.

For the purposes of the present invention, the term “filler” refers to organic and polymeric solid particles.

The fillers according to the invention participate in the dissolution or breakdown of the anhydrous solid composition of the invention, in particular in the presence of water. They may also contribute towards improving the cosmetic performance qualities due to the other compounds present in the composition.

Certain fillers may also have “anticaking” properties.

The polymeric organic filler(s) present in the composition of the invention are preferably chosen from polysaccharides and mixtures thereof.

In particular, the polymeric organic fillers are preferably chosen from non-cationic polysaccharides and mixtures thereof.

Mention may be made in particular of cyclodextrins, starches, alginates, gellans, guar gums, celluloses and wood flours. Among the polymeric organic fillers, mention may also be made of crosslinked polyvinylpyrrolidones and polyacrylates (for example Aquakeep).

Preferably, the polymeric organic filler(s) are chosen from cyclodextrins, starches, alginates, gellans, guar gums, celluloses, wood flours, crosslinked polyvinylpyrrolidones, polyacrylates and mixtures thereof, more preferentially from celluloses, starches and mixtures thereof, and better still from starches and mixtures thereof.

The total content of the polymeric organic filler(s) present in the anhydrous solid composition according to the invention is preferably greater than or equal to 20% by weight, more preferentially greater than or equal to 30% by weight and better still greater than or equal to 35% by weight, relative to the total weight of the composition.

Advantageously, the total content of the polymeric organic filler(s) present in the anhydrous solid composition according to the invention ranges from 20% to 80% by weight, preferably from 30% to 70% by weight and more preferentially from 35% to 60% by weight relative to the total weight of the composition.

In a preferred embodiment, the polymeric organic filler(s) are chosen from starches and mixtures thereof, and the total content of the starch(es) present in the anhydrous solid composition according to the invention is preferably greater than or equal to 20% by weight, more preferentially greater than or equal to 30% by weight, better still greater than or equal to 35% by weight, relative to the total weight of the composition.

According to this embodiment, advantageously, the total content of the starch(es) present in the anhydrous solid composition according to the invention ranges from 20% to 80% by weight, preferably from 30% to 70% by weight and more preferentially from 35% to 60% by weight, relative to the total weight of the composition.

The Cationic Polymers

The anhydrous solid composition according to the present invention may optionally also comprise one or more cationic polymers other than the polymeric organic fillers defined above.

For the purposes of the present invention, the term “cationic polymer” means any polymer comprising cationic groups and/or groups that may be ionized into cationic groups. Preferably, the cationic polymer(s) are hydrophilic or amphiphilic.

The cationic polymers are preferably not silicone-based (they do not comprise any Si—O units).

The preferred cationic polymers are chosen from those that contain units including primary, secondary, tertiary and/or quaternary amine groups that may either form part of the main polymer chain or may be borne by a side substituent directly connected thereto.

Preferably, the cationic polymers according to the invention do not comprise any anionic groups or any groups that can be ionized into anionic groups.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:
(1) homopolymers or copolymers derived from acrylic or methacrylic esters or amides and including at least one of the units having the following formulae:

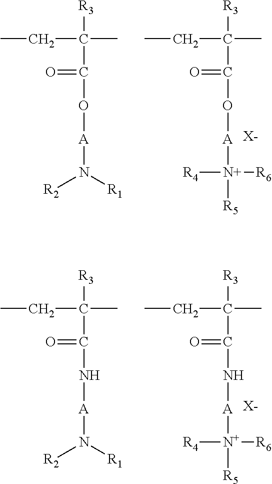

in which formulae:
   R₃, which may be identical or different, denote a hydrogen atom or a CH₃ radical;
   A, which may be identical or different, represent a linear or branched divalent alkyl group of 1 to 6 carbon atoms, preferably 2 or 3 carbon atoms, or a hydroxyalkyl group of 1 to 4 carbon atoms;
   R₄, R₅ and R₆, which may be identical or different, represent an alkyl group containing from 1 to 18 carbon atoms or a benzyl radical, and preferably an alkyl group containing from 1 to 6 carbon atoms;
   R₁ and R₂, which may be identical or different, represent a hydrogen atom or an alkyl group containing from 1 to 6 carbon atoms, preferably methyl or ethyl; and
   X denotes an anion derived from a mineral or organic acid, such as a methosulfate anion or a halide such as chloride or bromide.

The copolymers of the family (1) may also contain one or more units deriving from comonomers which may be chosen from the family of acrylamides, methacrylamides, diacetone acrylamides, acrylamides and methacrylamides substituted on the nitrogen with lower alkyls (C₁-C₄), acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by the company ISP;
polymers, preferably crosslinked polymers, of methacryloyloxy (C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (VI):

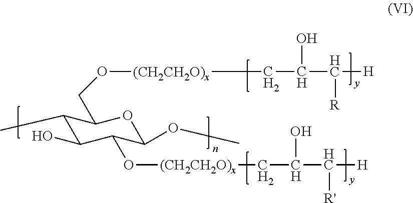

in which:
R represents an ammonium group RaRbRcN⁺—, Q⁻ in which Ra, Rb and Rc, which may be identical or different, represent a hydrogen atom or a linear or branched C₁ to C₃₀ alkyl, preferably an alkyl, and Q⁻ represents an anionic counterion such as a halide, for instance a chloride or bromide;
R′ represents an ammonium group R′aR′bR′cN⁺—, Q′⁻ in which R′a, R′b and R′c, which may be identical or different, represent a hydrogen atom or a linear or branched C₁ to C₃₀ alkyl, preferably an alkyl, and Q′ represents an anionic counterion such as a halide, for instance a chloride or bromide;
it being understood that at least one of the radicals Ra, Rb, Rc, R′a, R′b and R′c represents a linear or branched C₈ to C₃₀ alkyl;
n, x and y, which may be identical or different, represent an integer between 1 and 10 000.

Preferably, in formula (VI), at least one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (VI), only one of the radicals Ra, Rb, Rc, R′a, R′b or R′c represents a linear or branched C₈ to C₃₀, better still C₁₀ to C₂₄ or even C₁₀ to C₁₄ alkyl; mention may be made in particular of the dodecyl radical (C₁₂). Preferably, the other radicals represent a linear or branched C₁ to C₄ alkyl, notably methyl.

Better still, R may be a group chosen from —N⁺(CH₃)₃, Q′⁻ and —N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200©, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (VI) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂ (C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (VI) are, for example, those whose viscosity is between 2000 and 3000 cPs inclusive, preferentially between 2700 and 2800 cPs (between 2.7 and 2.8 Pa·s). Typically, Softcat Polymer SL-5 has a viscosity of 2500 cPs (2.5 Pa·s), Softcat Polymer SL-30 has a viscosity of 2700 cPs, Softcat Polymer SL-60 has a viscosity of 2700 cPs (2.7 Pa·s) and Softcat Polymer SL-100 has a viscosity of 2800 cPs (2.8 Pa·s). Use may also be made of Softcat Polymer SX-1300X with a viscosity of between 1000 and 2000 cPs (between 1 and 2 Pa·s).

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17 and Jaguar C162 by the company Rhodia.
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.
(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they include one or more tertiary amine functions, they can be quaternized;
(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.
(7) cyclopolymers of alkyldiallylamine or of dialkyldiallylammonium, such as homopolymers or copolymers including, as main constituent of the chain, units corresponding to formula (VII) or (VIII):

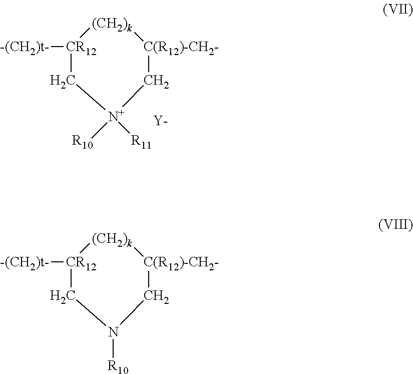

in which formulae (VII) and (VIII):
k and t are equal to 0 or 1, the sum k+t being equal to 1;
R₁₂ denotes a hydrogen atom or a methyl radical;
R₁₀ and R₁₁, independently of each other, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR.
(8) quaternary diammonium polymers comprising repeating units of formula (IX):

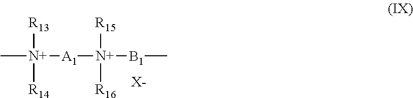

in which formula (IX):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ can also denote a group (CH₂)ₙCO-D-OC—(CH₂)ₙ— in which D denotes:
   a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
   d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers consisting of repeating units corresponding to the formula (X):

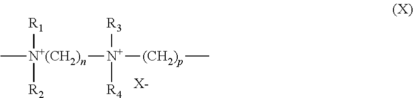

in which formula (X) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (X) that is particularly preferred is the one for which R₁, R₂, R₃ and R₄ represent a methyl radical and n=3, p=6 and X=Cl, which is known as Hexadimethrine chloride according to the INCI (CTFA) nomenclature.
(9) polyquaternary ammonium polymers comprising units of formula (XI):

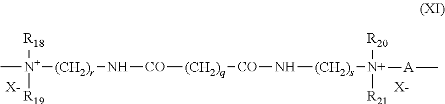

in which formula (XI):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.
(10) quaternary polymers of vinylpyrrolidone and of vinylimidazole, for instance the products sold under the names Luviquat® FC 905, FC 550 and FC 370 by the company BASF.
(11) polyamines such as Polyquart® H sold by Cognis, which is referenced under the name Polyethylene Glycol (15) Tallow Polyamine in the CTFA dictionary.
(12) polymers including in their structure:
(a) one or more units corresponding to formula (A) below:

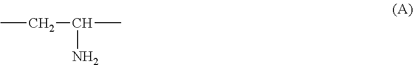

(b) optionally one or more units corresponding to formula (B) below:

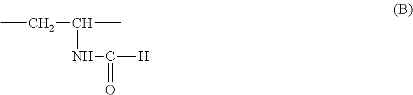

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The cationic charge density of these polymers may range from 2 meq/g to 20 meq/g, preferably from 2.5 to 15 meq/g and more particularly from 3.5 to 10 meq/g.

The polymers including units of formula (A) and optionally units of formula (B) are notably sold under the name Lupamin by the company BASF, for instance, in a non-limiting manner, the products sold under the names Lupamin 9095, Lupamin 5095, Lupamin 1095, Lupamin 9030 (or Luviquat 9030) and Lupamin 9010.

Preferably, the cationic polymer(s) are chosen from cationic polysaccharides (family (2)) and mixtures thereof, more preferentially from cationic galactomannan gums and mixtures thereof, and better still from cationic guar gums and mixtures thereof.

The total content of the cationic polymer(s), when they are present in the anhydrous solid composition according to the invention, is preferably greater than or equal to 0.05% by weight; more preferentially this total content ranges from 0.05% to 5% by weight, better still from 0.1% to 2% by weight, and even more preferentially from 0.2% to 1.5% by weight, relative to the total weight of the composition.

According to a preferred embodiment, the cationic polymer(s) are chosen from polysaccharides and mixtures thereof, and the total content of the cationic polysaccharide(s) present in the anhydrous solid composition according to the invention is preferably greater than or equal to 0.05% by weight, more preferentially ranging from 0.05% to 5% by weight, better still from 0.1% to 2% by weight, or even from 0.2% to 1.5% by weight, relative to the total weight of the composition.

The Nonionic Surfactants

The anhydrous solid composition according to the present invention may optionally further comprise one or more nonionic surfactants.

Examples of nonionic surfactants which can be used in the compositions of the present invention are described for example in “Handbook of Surfactants” by M. R. Porter, published by Blackie & Son (Glasgow and London), 1991, pp. 116-178. They are chosen notably from alcohols, alpha-diols, alkyl(C₁-C₂₀) phenols or fatty acids, these compounds being polyethoxylated, polypropoxylated or polyglycerolated, and having at least one fatty chain containing, for example, from 8 to 18 carbon atoms, it being possible for the number of ethylene oxide or propylene oxide groups to range notably from 1 to 100 and for the number of glycerol groups to range notably from 1 to 30.

Mention may also be made of condensates of ethylene oxide and propylene oxide with fatty alcohols; polyethoxylated fatty amides having preferably from 1 to 30 ethylene oxide units, polyglycerolated fatty amides containing on average from 1 to 5 glycerol groups and more particularly from 1.5 to 4, ethoxylated sorbitan fatty acid esters having from 2 to 30 ethylene oxide units, sucrose fatty acid esters, polyethylene glycol fatty acid esters, (C₆ to C₂₄ alkyl)polyglycosides, N—(C₆ to C₂₄ alkyl)glucamine derivatives, and amine oxides such as (C₁₀ to C₁₄ alkyl)amine oxides or N—(C₁₀ to C₁₄ acyl)aminopropylmorpholine oxides.

Among the nonionic surfactants, preference is given more particularly to using alkyl(poly)glycoside nonionic surfactants.

The term “alkyl(poly)glycoside” denotes an alkylpolyglycoside or an alkylmonoglycoside, also called alkylglycoside in the present patent application, which may be alkoxylated by one or more preferably C₂ to C₄ alkylene oxide groups.

The alkyl(poly)glycoside nonionic surfactant(s), used alone or in (a) mixture(s), in accordance with the present invention may be represented by the formula (XII) below:

R₁O—(R₂O)ₜ(G)ᵥ (XII)

formula (XII), in which:
R₁ represents a saturated or unsaturated, linear or branched alkyl group containing from 8 to 24 carbon atoms or an alkylphenyl group in which the linear or branched alkyl group contains from 8 to 24 carbon atoms,
R₂ represents an alkylene group containing approximately from 2 to 4 carbon atoms,
G represents a saccharide unit containing from 5 to 6 carbon atoms,
t denotes a value ranging from 0 to 10, preferably 0 to 4, and
v denotes a value ranging from 1 to 15.

The alkyl(poly)glycoside nonionic surfactant(s) preferably conform to the formula (XII) in which:
R₁ denotes a saturated or unsaturated, linear or branched alkyl group containing from 8 to 18 carbon atoms,
G denotes glucose, fructose or galactose, and preferably glucose,
t denotes a value ranging from 0 to 3, and is preferably equal to 0, and
R₂ and v are as defined above.

The degree of polymerization of the alkyl(poly)glycoside nonionic surfactant(s), as represented for example by the index v in the formula (XII) above, varies on average from 1 to 15, and preferably from 1 to 4. This degree of polymerization varies more particularly from 1 to 2, and even better still from 1.1 to 1.5, on average.

The glycoside bonds between the saccharide units are 1,6 or 1,4, and preferably 1,4.

The alkyl(poly)glycoside nonionic surfactants which may be used in the present invention are preferably alkyl(poly)glucosides notably represented by the products sold by Cognis under the names Plantaren® (600 CS/U, 1200 and 2000) or Plantacare® (818, 1200 and 2000). Use may also be made of the products sold by Seppic under the names Triton CG 110 (or Oramix CG 110) and Triton CG 312 (or Oramix® NS 10), the products sold by BASF under the name Lutensol GD 70 or else those sold by Chem Y under the name AG10 LK, or the products sold by Evonik Goldschmidt under the trade names Tego Care CG 90 or Tego Care GC 90 MB.

As nonionic surfactant it is possible with preference to use the compounds with INCI name caprylyl/capryl glucoside, decyl glucoside, Coco glucoside, lauryl glucoside, myristyl glucoside, cetearyl glucoside and/or arachidyl glucoside. The compound with INCI name cetearyl glucoside is particularly preferred.

The total content of the nonionic surfactant(s), when they are present in the anhydrous solid composition according to the invention, ranges preferably from 0.1% to 10% by weight, and more preferentially from 0.2% to 5% by weight, relative to the total weight of the composition.

Cationic Surfactants

The anhydrous solid composition according to the present invention may optionally also comprise one or more cationic surfactants.

The term “cationic surfactant” means a surfactant that is positively charged when it is contained in the compositions according to the invention. This surfactant may bear one or more positive permanent charges or may contain one or more cationizable functions within the compositions according to the invention.

The cationic surfactants are advantageously chosen from optionally polyoxyalkylenated primary, secondary or tertiary fatty amine salts, quaternary ammonium salts, and mixtures thereof.

As quaternary ammonium salts, mention may notably be made of:
the quaternary ammonium salts of formula (XIII):

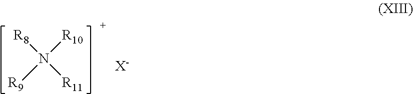

in which:
the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 and preferably from 12 to 24 carbon atoms, it being possible for the aliphatic groups to include heteroatoms notably such as oxygen, nitrogen, sulfur and halogens; and
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.

The aliphatic groups R₈ to R₁₁ may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido (C₂-C₆)alkyl, (C₁₂-C₂₂)alkyl acetate, and C₁-C₃₀ hydroxyalkyl groups.

Mention may notably be made of tetraalkylammonium halides, notably chlorides, such as dialkyldimethylammonium or alkyltrimethylammonium chlorides in which the alkyl group includes from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and notably chlorides; notably the product sold under the name Ceraphyl® 70 by the company Van Dyk.
the quaternary ammonium salts of imidazoline of formula (XIV):

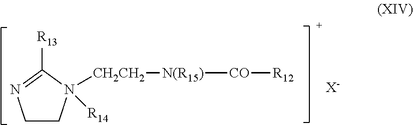

in which:
R₁₂ represents an alkenyl or alkyl group including from 8 to 30 carbon atoms, for example tallow fatty acid derivatives;
R₁₃ represents a hydrogen atom, a C₁ to C₄ alkyl group or an alkenyl or alkyl group including from 8 to 30 carbon atoms;
R₁₄ represents a C₁ to C₄ alkyl group;
R₁₅ represents a hydrogen atom or a C₁ to C₄ alkyl group; and
X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.

Preferably, R₁₂ and R₁₃ denote a mixture of alkenyl or alkyl groups including from 12 to 21 carbon atoms, for example tallow fatty acid derivatives, R₁₄ denotes a methyl group and R₁₅ denotes a hydrogen atom. Such a product is sold, for example, under the name Rewoquat® W75 or W90 by the company Evonik.
the quaternary di- or triammonium salts of formula (XV):

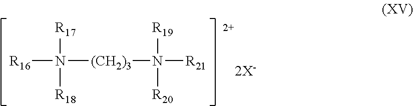

in which:
R₁₆ denotes an alkyl group including from 16 to 30 carbon atoms, which is optionally hydroxylated and/or optionally interrupted with one or more oxygen atoms,
R₁₇ denotes hydrogen, an alkyl group including from 1 to 4 carbon atoms or a group —(CH₂)₃—N⁺(R₁₆ₐ)(R₁₇ₐ)(R₁₈ₐ), R₁₆ₐ, R₁₇ₐ and R₁₈ₐ, which may be identical or different, denoting hydrogen or an alkyl group including from 1 to 4 carbon atoms,
R₁₈, R₁₉, R₂₀ and R₂₁, which may be identical or different, denote hydrogen or an alkyl group including from 1 to 4 carbon atoms, and
X⁻ is an anion, chosen notably from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P (Quaternium 89) and Finquat CT (Quaternium 75), sold by the company Finetex;
quaternary ammonium salts containing one or more ester functions, of formula (XVI) below:

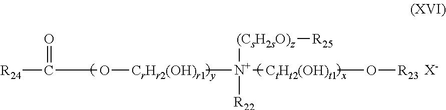

in which:
R₂₂ is chosen from C₁ to C₆ alkyl and C₁ to C₆ hydroxyalkyl or dihydroxyalkyl groups;
R₂₃ is chosen from the group R₂₆—C(═O)—, linear or branched, saturated or unsaturated C₁ to C₂₂ hydrocarbon-based groups R₂₇, and a hydrogen atom;
R₂₅ is chosen from the group R₂₈—C(═O)—, linear or branched, saturated or unsaturated C₁ to C₆ hydrocarbon-based groups R₂₉, and a hydrogen atom;
R₂₄, R₂₆ and R₂₈, which may be identical or different, are chosen from linear or branched, saturated or unsaturated C₇ to C₂₁ hydrocarbon-based groups;
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻ is an anion,
it being understood that r2+r1=2r and t1+t2=2t, and that the sum x+y+z ranges from 1 to 15,
with the proviso that when x=0 then R₂₃ denotes R₂₇ and that when z=0 then R₂₅ denotes R₂₉.

The alkyl groups R₂₂ may be linear or branched, preferably linear. Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may comprise from 12 to 22 carbon atoms, or else may comprise from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁ to C₂₁ hydrocarbon-based groups, and more particularly from linear or branched C₁₁ to C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate, a (C₁-C₄))alkylsulfonate or a (C₁-C₄)alkylarylsulfonate, a methanesulfonate, a phosphate, a nitrate, a tosylate, an anion derived from organic acid such as an acetate or a lactate or any other anion that is compatible with the ammonium bearing an ester function. The anion X⁻ is more particularly a chloride, a methyl sulfate or an ethyl sulfate.

Use is more particularly made, in the composition according to the invention, of the ammonium salts of formula (XVI) in which:
R₂₂ denotes a methyl or ethyl group,
x and y are equal to 1,
z is equal to 0 or 1,
r, s and t are equal to 2,
R₂₃ is chosen from the group R₂₆—C(═O)—; methyl, ethyl or C₁₄ to C₂₂ hydrocarbon-based groups, and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃ to C₁₇ hydrocarbon-based groups, and preferably from linear or branched, saturated or unsaturated C₁₃ to C₁₇ alkyl and alkenyl groups.

Advantageously, the hydrocarbon-based groups are linear.

Among the compounds of formula (XVI), mention may be made of the salts, notably the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or monoacyloxyethyl-hydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures notably of plant or animal origin, or by transesterification of the methyl esters thereof. This esterification may be followed by quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a weight majority of diester salts. Use may also be made of the ammonium salts containing at least one ester function that are described in patents U.S. Pat. Nos. 4,874,554 and 4,137,180. Use may also be made of the behenoylhydroxypropyltrimethylammonium chloride sold, for example, by the company Kao under the name Quartamin BTC 131.

Preferably, the ammonium salts containing at least one ester function contain two ester functions.

Advantageously, when they are present in the anhydrous solid composition according to the invention, the content of the cationic surfactant(s) ranges from 0.01% to 5% by weight, preferably from 0.05% to 3% by weight, and more preferentially from 0.1% to 2% by weight, relative to the total weight of the composition.

The total content of surfactants present in the anhydrous solid composition according to the invention is preferably less than or equal to 60% by weight; more preferentially this total content ranges from 20% to 55% by weight, even better still from 30% to 50% by weight, and better still even from 35% to 45% by weight, relative to the total weight of the composition.

The Organic Solvents

The anhydrous solid composition according to the present invention may optionally further comprise one or more organic solvents.

Preferably, the organic solvent(s) are chosen from linear or branched monoalcohols containing from 1 to 8 carbon atoms, and more preferentially from 1 to 4 carbon atoms, polyols, notably C₂ to C₈ polyols, polyethylene glycols, aromatic alcohols, and mixtures thereof.

As examples of organic solvents that may be used according to the invention, mention may notably be made of ethanol, propanol, butanol, isopropanol, isobutanol, propylene glycol, dipropylene glycol, isoprene glycol, butylene glycol, glycerol, benzyl alcohol and phenoxyethanol, and mixtures thereof.

The organic solvent(s) that may be used according to the invention may be chosen from linear or branched monoalcohols containing from 1 to 4 carbon atoms, and mixtures thereof, preferably from ethanol, propanol, butanol, isopropanol, isobutanol, and mixtures thereof.

Preferably, the organic solvent(s) are chosen from polyols, notably C₂ to C₈ polyols, and mixtures thereof, and more preferentially from glycerol, propylene glycol, and mixtures thereof.

In a preferred embodiment of the invention, the anhydrous solid composition comprises one or more organic solvents, preferentially one or more polyols.

The total content of the organic solvent(s), when they are present in the anhydrous solid composition according to the invention, is preferably less than or equal to 20% by weight, more preferentially less than or equal to 15% by weight, and better still ranges from 0.5% to 12% by weight, relative to the total weight of the composition.

The anhydrous solid composition according to the present invention may optionally further comprise one or more additional compounds, different from the compounds defined above, and preferably chosen from anionic and nonionic polymers and mixtures thereof, antioxidants, penetrants, sequestrants, fragrances, buffers, dispersants, conditioning agents such as, for example, volatile or non-volatile, modified or non-modified silicones, film formers, ceramides, preservatives, opacifiers, lubricants (or anti-caking agents) and mixtures thereof.

According to a particular embodiment, the anhydrous solid composition of the invention may optionally comprise one or more lubricants. Such a lubricant may notably act as an anti-caking agent.

The lubricant(s) which may be used are different from the polymeric organic fillers and the cationic polymers defined above.

The lubricants which may be used in the anhydrous solid composition of the invention notably include silica, more particular anhydrous colloidal silica, sericite, polyamide (Nylon®) powders, poly-p-alanine and polyethylene powders, powders of tetrafluoroethylene polymers (Teflon®), acrylate-dimethicone copolymers, stearic acid, metal soaps derived from organic carboxylic acids having from 8 to 22 carbon atoms, preferably from 12 to 18 carbon atoms, such as, for example, zinc, magnesium or lithium stearates, zinc laurate and magnesium myristate, alkali metal or alkaline-earth metal carbonates, such as, for example, magnesium, sodium and calcium carbonates, fatty acids such as stearic acid, celluloses, notably crystalline celluloses, and mixtures thereof.

According to one embodiment, the lubricant or lubricants are advantageously chosen from metal soaps derived from organic carboxylic acids having from 8 to 22 carbon atoms, preferably from 12 to 18 carbon atoms, and mixtures thereof, even better still from zinc stearate, magnesium stearate, lithium stearate, zinc laurate, magnesium myristate and mixtures thereof. The lubricant more preferentially is magnesium stearate.

According to another embodiment, the lubricant or lubricants are advantageously chosen from alkali metal or alkaline-earth metal carbonates and mixtures thereof, preferably from magnesium carbonate, sodium carbonate, calcium carbonate and mixtures thereof; and more preferentially magnesium carbonate.

Preferably, when the additional compound or compounds above are present in the anhydrous solid composition according to the invention, the additional compound or compounds are present in general in an amount each of between 0.01% and 20% by weight, relative to the weight of the composition.

Needless to say, a person skilled in the art will take care to select this or these optional additional compounds such that the advantageous properties intrinsically associated with the anhydrous solid composition of the invention are not, or are not substantially, adversely affected by the envisaged addition(s).

A subject of the present invention is also a cosmetic treatment process, and notably a process for washing and/or conditioning keratin fibres, in particular human keratin fibres such as the hair, comprising the application to said keratin fibres of a solid composition as defined previously, the solid composition being applied directly to said keratin fibres or after having been moistened beforehand with water.

The anhydrous solid composition according to the invention may be applied to dry or wet keratin fibres, preferably to wet keratin fibres.

The anhydrous solid composition thus applied may optionally be rinsed off or left on, after an optional leave-on time that may range from 1 to 15 minutes, preferably from 2 to 10 minutes.

Preferably, the anhydrous solid composition is rinsed off after application.

According to a first embodiment of the invention, the anhydrous solid composition is applied directly to the keratin fibres, i.e. without being moistened and/or broken down in water beforehand.

When, according to this first embodiment, the anhydrous solid composition of the invention is applied directly (i.e. without being moistened or broken down beforehand) to the dry keratin fibres, water may optionally be added to said fibres in order subsequently to rub/massage so as to dissolve/pre-emulsify said composition and to form an immediate abundant foam. The foam thus obtained can subsequently be rinsed out after an optional leave-on time.

Conversely, the anhydrous solid composition of the invention may also be applied directly (i.e. without moistening or breaking down beforehand) to the wet keratin fibres, followed by massaging/rubbing to break down the particles and to obtain an immediate abundant foam. The foam thus obtained can subsequently be rinsed out after an optional leave-on time.

According to another embodiment of the invention, the anhydrous solid composition is moistened and/or broken down beforehand in water before being applied to the keratin fibres. According to this embodiment, a small amount (preferably ranging from 1 to 3 g) of anhydrous solid composition is advantageously taken up and dissolved with water, for example in the hand, so as to form an immediate abundant foam. The foam thus obtained may then be applied to the wet or dry keratin fibres, before being optionally rinsed out with water after an optional leave-on time.

A subject of the present invention is also the use of a solid composition as defined previously for washing and/or conditioning keratin fibres, in particular human keratin fibres such as the hair.

The present invention also relates to a packaging article, preferably a cosmetic packaging article, comprising:
an envelope defining at least one cavity, the envelope comprising one or more water-soluble and/or liposoluble compounds;
a solid composition as defined above;
it being understood that the solid composition is in one of the cavities defined by the envelope.

The term “cosmetic packaging article” means an article that is suitable for cosmetic use; in particular for use of the packaging article on keratin fibres, notably the hair, and/or on the scalp. In particular, the packaging article makes it possible to wash and/or condition the keratin fibres, in particular human keratin fibres such as the hair.

Preferably, the packaging article according to the invention is water-soluble or liposoluble at a temperature of less than or equal to 35° C.

Preferably, the envelope of the packaging article according to the invention is water-soluble at a temperature of less than or equal to 35° C.

The term “water-soluble” means soluble in water, in particular in a proportion of at least 10 grams per litre of water, preferably at least 20 g/l, better still at least 50 g/l, at a temperature of less than or equal to 35° C. Thus, when water preferably having a temperature of less than 35° C. is added to the packaging article, the envelope dissolves and releases the anhydrous solid composition present in one of the cavities of the envelope.

The term “liposoluble” means soluble in a liquid fatty substance as defined below, in particular in a proportion of at least 10 grams per litre of liquid fatty substance, in particular in a plant or mineral oil such as liquid petroleum jelly, preferably at least 20 g/l in a liquid fatty substance, better still at least 50 g/l in a fatty substance, at a temperature of less than or equal to 35° C.

The term “temperature of less than or equal to 35° C.” means a temperature not exceeding 35° C. but greater than or equal to 0° C., for example ranging from more than 1 to 35° C., preferably from 5 to 30° C., more preferentially from 10 to 30° C. and better still from 15 to 25° C. It is understood that all the temperatures are given at atmospheric pressure (1 atm).

The packaging article may comprise one or more cavities, at least one of which contains the anhydrous solid composition as defined previously. Preferably, the packaging article comprises only one cavity in which the anhydrous solid composition is contained.

Advantageously, the envelope represents from 0.5% to 20% by weight, preferably from 1% to 15% by weight, more preferentially from 2% to 10% by weight and better still from 4% to 8% by weight relative to the total weight of the packaging article.

Advantageously, the anhydrous solid composition as defined previously represents from 80% to 99.5% by weight, preferably from 85% to 99% by weight, more preferentially from 90% to 98% by weight and better still from 92% to 96% by weight relative to the total weight of the packaging article.

The weight ratio between the total weight of the anhydrous solid composition of the invention and the total weight of the envelope advantageously ranges from 80/20 to 99/1, preferably from 85/15 to 98/2 and more preferentially from 90/10 to 97/3.

The envelope of the packaging article comprises one or more water-soluble and/or liposoluble compounds, preferably one or more water-soluble compounds advantageously chosen from water-soluble polymers and mixtures thereof.

The water-soluble polymer(s) that may be used according to the present invention contain water-soluble units in their backbones. The water-soluble units are obtained from one or more water-soluble monomers.

The term “water-soluble monomer” means a monomer whose solubility in water is greater than or equal to 1%, preferably greater than or equal to 5%, at 25° C. and at atmospheric pressure (760 mmHg).

Said water-soluble polymer(s) that are capable of forming the envelope are advantageously obtained from water-soluble monomers including at least one double bond. These monomers may be chosen from cationic, anionic and nonionic monomers, and mixtures thereof.

As water-soluble monomers that may be used as precursors for the water-soluble units, alone or as a mixture, examples that may be mentioned include the following monomers, which may be in free or salified form:
(meth)acrylic acid,
styrenesulfonic acid,
vinylsulfonic acid and (meth)allylsulfonic acid,
vinylphosphonic acid,
N-vinylacetamide and N-methyl-N-vinylacetamide,
N-vinylformamide and N-methyl-N-vinylformamide,
N-vinyllactams including a cyclic alkyl group containing from 4 to 9 carbon atoms, such as N-vinylpyrrolidone, N-butyrolactam and N-vinylcaprolactam,
maleic anhydride,
itaconic acid,
vinyl alcohol of formula CH₂═CHOH,
vinyl acetate of formula CH₂═CHOC(O)CH₃,
vinyl ethers of formula CH₂═CHOR in which R is a linear or branched, saturated or unsaturated hydrocarbon-based radical containing from 1 to 6 carbon atoms;
dimethyldiallylammonium halides (chloride),
quaternized dimethylaminomethyl methacrylate (DMAEMA),
(meth)acrylamidopropyltrimethylammonium halides (chloride) (APTAC and MAPTAC),
methylvinylimidazolium halides (chloride),
2-vinylpyridine and 4-vinylpyridine,
acrylonitrile,
glycidyl (meth)acrylate,
vinyl halides (chloride) and vinylidene chloride,
the vinyl monomers having the following formula: H₂C═C(R)—C(O)—X, in which:
R is chosen from H, (C₁-C₆)alkyl such as methyl, ethyl and propyl, and
X is chosen from:
   alkoxy groups of the type —OR′ in which R′ is a linear or branched, saturated or unsaturated hydrocarbon-based radical containing from 1 to 6 carbons, optionally substituted with at least one halogen (iodine, bromine, chlorine or fluorine); a group from among sulfonic (—SO₃⁻), sulfate (SO₄⁻), phosphate (—PO₄H₂); hydroxyl (—OH); primary amine (—NH₂); secondary amine (NHR₆), tertiary amine (—NR₆R₇) or quaternary amine (—N⁺R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6;
   groups —NH₂, —NHR′ and —NR′R″ in which R′ and R″ are, independently of each other, linear or branched, saturated or unsaturated hydrocarbon-based radicals containing from 1 to 6 carbons, with the proviso that the total number of carbon atoms of R′+R″ does not exceed 6, said radicals R′ and R″ being optionally substituted with a halogen (iodine, bromine, chlorine or fluorine); a group from among hydroxyl (—OH); sulfonic (—SO₃⁻), sulfate (SO₄⁻), phosphate (—PO₄H₂); primary amine (—NH₂); secondary amine (NHR₆), tertiary amine (—NR₆R₇) and/or quaternary amine (—N⁺R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6. As compounds corresponding to this formula, examples that may be mentioned include N,N-dimethylacrylamide and N,N-diethylacrylamide;
and mixtures thereof.

Anionic monomers that may notably be mentioned include (meth)acrylic acid, acrylamido-2-methylpropanesulfonic acid, itaconic acid and the salts thereof with an alkali metal, an alkaline-earth metal or ammonium or those derived from an organic amine such as an alkanolamine.

Nonionic monomers that may notably be mentioned include (meth)acrylamide, N-vinylformamide, N-vinylacetamide and hydroxypropyl (meth)acrylate, vinyl alcohol of formula CH₂═CHOH, and vinyl acetate of formula CH₂═CHOC(O)CH₃.

The cationic monomers are preferably chosen from quaternary ammonium salts derived from a diallylamine, and those corresponding to the following formula:

H₂C═C(R₁)-D-N⁺R₂R₃R₄,X⁻

in which:
R₁ represents a hydrogen atom or a methyl group,
R₂ and R₃, which may be identical or different, represent a hydrogen atom or a linear or branched C₁ to C₄ alkyl group,
R₄ represents a hydrogen atom, a linear or branched C₁-C₄ alkyl group or an aryl group,
D represents the following divalent unit: —(Y)ₙ-(A)- in which:
   Y represents an amide function, an ester (O—C(O) or C(O)—O), a urethane or a urea,
   A represents a linear or branched, cyclic or acyclic C₁ to C₁₀ alkylene group, which may be substituted or interrupted with a divalent aromatic or heteroaromatic group. The alkylene groups may be interrupted with an oxygen atom, a nitrogen atom, a sulfur atom or a phosphorus atom; the alkylene possibly being interrupted with a ketone function, an amide, an ester (O—C(O) or C(O)—O), a urethane or a urea,
   n is an integer ranging from 0 to 1,
X⁻ represents an anionic counterion, for instance a chloride or a sulfate.

Examples of water-soluble cationic monomers that may notably be mentioned include the following compounds, and also the salts thereof: dimethylaminoethyl (meth)acrylate, (meth)acryloyloxyethyltrimethylammonium (meth)acrylate, (meth)acryloyloxyethyldimethylbenzylammonium (meth)acrylate, N-[dimethylaminopropyl](meth)acrylamide (meth)acrylate, (meth)acrylamidopropyltrimethylammonium (meth)acrylate, (meth)acrylamidopropyldimethylbenzyl-ammonium (meth)acrylate, dimethylaminohydroxypropyl (meth)acrylate, (meth)acryloyloxyhydroxypropyltrimethylammonium (meth)acrylate, (meth)acryloyl-oxyhydroxypropyldimethylbenzylammonium (meth)acrylate and dimethyldiallylammonium (meth)acrylate.

Among the water-soluble polymers that may be used according to the present invention, mention may also be made of polyhydroxyalcohol (PHA).

Preferably, the water-soluble polymers are polymerized from one or more monomers chosen from vinyl alcohol of formula CH₂═CHOH, vinyl acetate of formula CH₂═CHOC(O)CH₃ and mixtures thereof.

The water-soluble polymers that are capable of forming the envelope of the packaging article may also be chosen from water-soluble polymers derived from natural products, such as polysaccharides, i.e. polymers bearing sugar units. These water-soluble polymers are different from the cationic polysaccharide(s) present in the anhydrous solid composition.

The term “sugar unit” means a unit derived from a carbohydrate of formula Cₙ(H₂O)ₙ₋₁ or (CH₂O)ₙ, which may be optionally modified by substitution and/or by oxidation and/or by dehydration. The sugar units that may be included in the composition of the polymers of the invention are preferably derived from the following sugars: glucose, galactose, arabinose, rhamnose, mannose, xylose, fucose, fructose, anhydrogalactose, galacturonic acid, glucuronic acid, mannuronic acid, galactose sulfate, anhydrogalactose sulfate.

The polymers bearing sugar unit(s) according to the invention may be of natural or synthetic origin. They may be nonionic, anionic, cationic or amphoteric. The base units of the polymers bearing a sugar unit of the invention may be monosaccharides or disaccharides.

As polymers that may be used, mention may notably be made of the following native gums, and also derivatives thereof:
a) tree or shrub exudates, including:
acacia gum (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (or tragacanth)(polymer of galacturonic acid, galactose, fucose, xylose and arabinose);
b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);
c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose), the main constituent of which is glucomannan, which is a polysaccharide of high molecular weight (500 000<M_{glucomannan}<2 000 000) composed of D-mannose and D-glucose units with a branch every 50 or 60 units approximately;
d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
biosaccharide gum (polymer of galacturonic acid, fucose and D-galactose),
e) plant extracts, including:
cellulose (glucose polymer);
starch (glucose polymer);
inulin (polymer of fructose and glucose).

These polymers may be physically or chemically modified. A physical treatment that may notably be mentioned is the temperature. Chemical treatments that may be mentioned include esterification, etherification, amidation and oxidation reactions. These treatments can lead to polymers that may be nonionic, anionic, cationic or amphoteric.

Preferably, these chemical or physical treatments are applied to guar gums, locust bean gums, starches and celluloses.

The nonionic guar gums that may be used according to the invention may be modified with C₁ to C₆ hydroxyalkyl groups. Among the hydroxyalkyl groups, mention may be made of hydroxymethyl, hydroxyethyl, hydroxypropyl and hydroxybutyl groups.

These guar gums are well known in the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The guar gums modified with cationic groups that may more particularly be used according to the invention are guar gums including trialkylammonium cationic groups. Preferably, 2% to 30% by number of the hydroxyl functions of these guar gums bear trialkylammonium cationic groups. Even more preferentially, 5% to 20% by number of the hydroxyl functions of these guar gums are branched with trialkylammonium cationic groups. Among these trialkylammonium groups, mention may most particularly be made of the trimethylammonium and triethylammonium groups. Even more preferentially, these groups represent from 5% to 20% by weight relative to the total weight of the modified guar gum.

According to the invention, guar gums modified with 2,3-epoxypropyltrimethylammonium chloride may be used.

These guar gums modified with cationic groups are products already known per se and are, for example, described in patents U.S. Pat. Nos. 3,589,578 and 4,013,307. Such products are moreover notably sold under the trade names Jaguar C13S, Jaguar C15 and Jaguar C17 by the company Rhodia Chimie.

As modified locust bean gum, use may be made of cationic locust bean gum containing hydroxypropyltrimonium groups, such as Catinal CLB 200 sold by the company Toho.

The starch molecules used in the present invention may originate from any plant source of starch, notably cereals and tubers; more particularly, they may be starches from corn, rice, cassava, barley, potato, wheat, sorghum, pea, oat or tapioca. It is also possible to use hydrolysates of the starches mentioned above. The starch is preferably derived from potato.

The starches may be chemically or physically modified, notably by one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents, leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus be bonded together (for example with glyceryl and/or phosphate groups);
esterification in alkaline medium for the grafting of functional groups, notably C₁ to C₆ acyl (acetyl), C₁ to C₆ hydroxyalkyl (hydroxyethyl or hydroxypropyl), carboxymethyl or octenylsuccinic.

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may notably be obtained by crosslinking with phosphorus compounds; with St meaning starch and X notably denoting alkali metals (for example sodium or potassium), alkaline-earth metals (for example calcium or magnesium), ammonia salts, amine salts such as salts of monoethanolamine, diethanolamine, triethanolamine or 3-amino-1,2-propanediol, and ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates or compounds rich in distarch phosphate may notably be mentioned, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetylated cassava distarch phosphate) by the company Avebe, or Structure Zea from National Starch (gelatinized corn distarch phosphate).

A preferred starch is a starch that has undergone at least one chemical modification such as at least one esterification.

According to the invention, use may also be made of amphoteric starches, comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic. The cationic groups may be of primary, secondary, tertiary or quaternary amine type.

The amphoteric starches are notably chosen from the compounds having the following formulae:

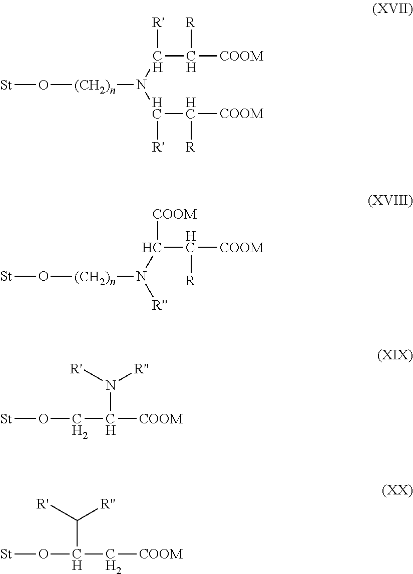

in which formulae (XVII) to (XX):
St-O represents a starch molecule;
R, which may be identical or different, represents a hydrogen atom or a methyl radical;
R′, which may be identical or different, represents a hydrogen atom, a methyl radical or a —C(O)—OH group;
n is an integer equal to 2 or 3;
M, which may be identical or different, denotes a hydrogen atom, an alkali metal or alkaline-earth metal such as Na, K or Li, a quaternary ammonium NH₄, or an organic amine; and
R″ represents a hydrogen atom or a C₁-C₁₈ alkyl radical.

These compounds are notably described in patents U.S. Pat. Nos. 5,455,340 and 4,017,460.

Starches of formula (XVIII) or (XIX), and preferentially starches modified with 2-chloroethylaminodipropionic acid are particularly used, i.e. starches of formula (XVIII) or (XIX) in which R, R′, R″ and M represent a hydrogen atom and n is equal to 2. Preferably, the amphoteric starch is a starch chloroethylamido dipropionate.

The celluloses and cellulose derivatives may be anionic, cationic, amphoteric or nonionic.

Among these derivatives, cellulose ethers, cellulose esters and cellulose ester ethers are distinguished.

Among the cellulose esters, mention may be made of inorganic esters of cellulose (cellulose nitrates, sulfates or phosphates), organic esters of cellulose (cellulose monoacetates, triacetates, amidopropionates, acetatebutyrates, acetatepropionates or acetatetrimellitates), and mixed organic/inorganic esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and also the sodium salts thereof.

Among the cationic cellulose ethers, mention may be made of crosslinked or non-crosslinked quaternized hydroxyethylcelluloses. The quaternizing agent may notably be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Among the associative polymers bearing sugar unit(s), mention may be made of celluloses or derivatives thereof, modified with groups including at least one fatty chain such as alkyl, arylalkyl or alkylaryl groups or mixtures thereof, in which the alkyl groups are C8-C22; nonionic alkylhydroxyethylcelluloses such as the products Natrosol Plus Grade 330 CS and Polysurf 67 (C₁₆ alkyl) sold by the company Aqualon; quaternized alkylhydroxyethylcelluloses (cationic) such as the products Quatrisoft LM 200, Quatrisoft LM-X 529-18-A, Quatrisoft LM-X5 29-18-B (C₁₂ alkyl) and Quatrisoft LM-X 529-8 (C₁₈ alkyl) sold by the company Amerchol, the products Crodacel QM, Crodacel QL (C₁₂ alkyl) and Crodacel QS (C₁₈ alkyl) sold by the company Croda and the product Softcat SL 100 sold by the company Amerchol; nonionic nonoxynylhydroxyethylcelluloses such as the product Amercell HM-1500 sold by the company Amerchol; nonionic alkylcelluloses such as the product Bermocoll EHM 100 sold by the company Berol Nobel.

As associative polymers bearing sugar unit(s) derived from guar, mention may be made of hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

The water-soluble polymer(s) bearing sugar unit(s) that may be used to form the envelope of the packaging article are preferably chosen from guar gums, locust bean gums, xanthan gums, starches and celluloses, in their modified (derived) form or unmodified form.

Preferably, said polymer(s) bearing sugar unit(s) are nonionic.

The water-soluble polymers described above more particularly have a weight-average molecular weight (Mw) of greater than 1 000 000 and preferably between 1 000000 and 50 000 000. The molecular weight is determined by the RSV (Reduced Specific Viscosity) method as defined in “Principles of Polymer Chemistry” Cornell University Press, Ithaca, N Y 1953 Chapter VII “Determination of Molecular Weight” pages 266-316.

The water-soluble or liposoluble compound(s) that are capable of forming the envelope of the packaging article according to the invention may be in fibre or film form.

According to a first embodiment, the water-soluble or liposoluble compound(s) are in the form of fibres. The term “fibre” refers to any object whose length is greater than its cross section. In other words, it should be understood as referring to an object of length L and of diameter D such that L is greater and preferably very much greater (i.e. at least three times greater) than D, D being the diameter of the circle in which the cross section of the fibre is inscribed. In particular, the ratio L/D (or aspect ratio) is chosen in the range extending from 3.5 to 2500, preferably from 5 to 500, and better still from 5 to 150. The cross section of a fibre may be of any shape: round, serrated or crenellated, or else bean-shaped, but also multilobal, in particular trilobal or pentalobal, X-shaped, in strip form, square, triangular, elliptical or the like. The fibres of the invention may or may not be hollow.

According to this embodiment, the fibres may be spun, carded or twisted. Advantageously, the fibres used in the context of the present invention are spun. The mean diameter of the fibres used according to the present invention, which may be identical or different, is less than 500 μm. Advantageously, such a diameter is less than 200 μm, preferably less than 100 μm, or even less than 50 μm.

Mention may be made more particularly of water-soluble fibres which include fibres based on PVA (polyvinyl alcohol), fibres of polysaccharides such as glucomannans, starches, celluloses such as carboxymethylcelluloses, polyalginic acid fibres, polylactic acid fibres and polyalkylene oxide fibres, and also mixtures thereof. More preferentially, the water-soluble fibre(s) used in the invention are chosen from PVA-based fibres.

The fibres of the envelope are generally entangled. The term “envelope comprising water-soluble fibres” means an envelope which may consist entirely of water-soluble fibres which may include both fibres that are water-soluble and fibres that are water-insoluble at a temperature of less than or equal to 35° C., the soluble fibres needing to be in larger amount than the insoluble fibres. The envelope of the fibres must include at least 60% by weight of soluble fibres, preferably at least 70% and better still at least 80% by weight relative to the total weight of the fibres. It may thus include, for example, more than 95% by weight, or even more than 99% by weight and even 100% by weight of water-soluble fibres relative to the total weight of the fibres of the envelope.

When the envelope contains insoluble fibres, these may be made of any material commonly used as insoluble fibres; they may be, for example, silk, cotton, wool, flax, polyamide (Nylon®), polylactic acid, modified cellulose (rayon, viscose, rayon acetate), poly-p-phenylene terephthalamide, notably Kevlar®, polyolefin and notably polyethylene or polypropylene, glass, silica, aramid, carbon, notably in graphite form, Teflon®, insoluble collagen, polyester, polyvinyl chloride or polyvinylidene chloride or polyethylene terephthalate fibres, or fibres formed from a mixture of the compounds mentioned above, such as polyamide/polyester or viscose/polyester fibres.

In addition, when the envelope contains fibres, it may be woven or nonwoven.

According to a first variant of the invention, the envelope may be woven. In the context of the present invention, a “woven” material results from an organized assembly of fibres, in particular of water-soluble polymeric fibres, and more particularly of an intercrossing, in the same plane, of said fibres, arranged in the direction of the warp and of fibres arranged, perpendicular to the warp fibres, in the direction of the weft. The bonding obtained between these warp and weft fibres is defined by a weave.

Such a woven material results from an operation directed towards assembling the fibres in an organized manner such as weaving per se, but may also result from knitting.

According to another variant of the invention, the envelope is nonwoven.

For the purposes of the present invention, the term “nonwoven fabric” refers to a substrate comprising fibres, in particular water-soluble polymeric fibres, in which the individual fibres are arranged in a disordered manner in a structure in the form of a lap and which are neither woven nor knitted. The fibres of the nonwoven fabric are generally bonded together, either under the effect of a mechanical action (for example needle punching, air jet or water jet), or under the effect of a thermal action, or by addition of a binder.

Such a nonwoven fabric is, for example, defined by the standard ISO 9092 as a web or lap of directionally or randomly oriented fibres, bonded by friction and/or cohesion and/or adhesion, excluding paper and products which are woven, knitted, tufted or stitch-bonded incorporating bonding yarns or filaments.

A nonwoven fabric differs from a paper by the length of the fibres used. In paper, the fibres are shorter. However, there are nonwoven fabrics based on cellulose fibre, which are manufactured by a wet-laid process and which have short fibres like in paper. The difference between a nonwoven fabric and a paper is generally the absence of hydrogen bonding between the fibres in a nonwoven fabric.

Very preferentially, the fibres used in the context of the present invention are chosen from synthetic fibres such as PVA fibres. In particular, the envelope is nonwoven, and is preferentially made of nonwoven PVA fibres.

To make the nonwoven envelope of the packaging article, use is preferably made of PVA fibres that are soluble in water at a temperature of less than or equal to 35° C., for instance the fibres sold by the Japanese company Kuraray under the name Kuralon K-II, and particularly the grade WN2 which is soluble at and above 20° C. These fibres are described in EP-A-636 716 which teaches the manufacture of PVA fibres that are soluble in water at temperatures not exceeding 100° C., by spinning and drawing of the wet or dry polyvinyl alcohol polymer in the presence of solvents participating in the dissolution and solidification of the fibre. The fibre thus obtained may lead to the production of woven or nonwoven substrates.

These fibres may also be prepared from a solution to be spun, by dissolving a water-soluble PVA-based polymer in a first organic solvent, spinning of the solution in a second organic solvent to obtain solidified filaments and wet drawing of the filaments, from which the first solvent is removed, followed by drying and subjecting to a heat treatment. The cross section of these fibres may be substantially circular. These fibres have a tensile strength of at least 2.7 g/dtex (3 g/d). Patent application EP-A-0 636 716 describes such water-soluble PVA-based fibres and the process for manufacturing them. For example, the fibres may also be formed by extrusion and deposited on a conveyor to form a lap of fibres which is then consolidated via a conventional fibre bonding technique, for instance needle punching, hot bonding, calendering or air-through bonding, in which technique the water-soluble lap passes through a tunnel into which hot air is blown, or spunlacing directed towards bonding the fibres under the action of fine jets of water at very high pressure, which cannot be applied to fibres whose dissolution temperature is too low.

As has been seen previously, the invention is not limited to the use of PVA, and use may also be made of fibres made from other water-soluble materials provided that these materials dissolve in water having the desired temperature, for example the polysaccharide fibres sold under the name Lysorb by the company Lysac Technologies Inc. or other fibres based on polysaccharide polymers such as glucomannans or starch.

The envelope may comprise a mixture of different fibres that are soluble in water at different temperatures (up to 35° C.).

The fibres may be composite, and they may include, for example, a core and a sheath which are not of the same nature, for example formed from different grades of PVA.

According to a particular embodiment of the invention, the envelope is a nonwoven fabric, including water-soluble fibres, alone or as a mixture with insoluble fibres as indicated above, with not more than 40% by weight of insoluble fibres relative to the total weight of the fibres constituting the lap. Preferably, the nonwoven fabric consists essentially of water-soluble fibres, i.e. it does not contain any insoluble fibres.

According to another embodiment of the invention, the envelope of the packaging article may consist of one or more films, which each comprise one or more water-soluble and/or liposoluble compounds, notably as defined above. When the envelope consists of several films, said films may be assembled, for example bonded together, so as to form a single unified film.

The thickness of the “overall” film (i.e. the thickness of the single film when the envelope contains only one or of the unified film when the envelope contains several films) is advantageously between 10 and 1000 microns, preferably between 10 and 800 microns and more preferentially between 15-500 microns.

The term “film” notably means a continuous layer preferentially formed from one or more water-soluble and/or liposoluble compounds as defined above, in particular of polymer(s).

The main industrial methods for the production of polymer films are extrusion of a molten polymer, casting of a solution of a polymer onto a polished metal surface (in certain cases, the polymer solution is introduced into a precipitation tank), casting of a dispersion of the polymer onto a polished surface, and calendering.

The films that may be used according to the present invention may be chosen from film-multilayer film, film-paper (laminating) and film-coating.

During application by spraying, brushing or various industrial processes, the surface coatings undergo what is known as the formation of a film, and notably of film-coating. In the majority of the film-forming processes, a liquid coating of relatively low viscosity is applied to a solid substrate and is hardened as a solid adherent film based on high molecular weight polymer having the properties desired by the user.

The films that may be used according to the present invention are notably PVA films which may be manufactured via any industrial production method, such as a method of casting a PVA-based polymer solution, a method of extrusion in the presence or absence of water, a dry-extrusion moulding method or a biaxial orientation method.

The packaging article, and the envelope, may have any shape that is suitable for the intended use, for example a rectangular, round or oval shape. Preferably, it has a rounded geometry, for example in the form of a sphere, a disc or an oval, or else a square or parallelepipedal geometry preferably with rounded corners. The envelope preferably has dimensions allowing it to be taken up between at least two fingers. Thus, it may, for example, have an ovoid shape about 2 to 10 cm long and about 0.5 to 4 cm wide, or a circular disc shape about 2 to 10 cm in diameter, or a square shape with a side length of about 2 to 15 cm, or a rectangular shape with a length of about 2 to 25 cm, it being understood that it may have any other shape and size that are suitable for the intended use.

Preferably, the envelope may be of round shape with an inside diameter ranging from 3 to 7 cm, more preferentially from 4 to 5 cm; to which may be added the dimension of the edges (sealed part) which may range from 1 to 5 mm, better still from 2 to 4 mm; and a height ranging from 2 to 7 mm, preferentially from 3 to 5 mm.

The envelope may also be of square or rectangular shape with a length preferably ranging from 2 to 6 cm, more preferentially from 3 to 5 cm, and a width preferably ranging from 2 to 5 cm, more preferentially 2.5 to 4 cm; to which may be added the dimension of the edges (sealed part) which may preferably range from 1 to 5 mm, and more preferentially from 2 to 4 mm.

Advantageously, the envelope has a low thickness, and may consist of several layers of different materials. Preferably, the thickness of the envelope ranges from 3% to 99.9% of its other dimensions. The envelope is thus substantially flat, with thin edge profiles.

The area delimiting the cavity or cavities has an extent advantageously less than 625 cm², preferably between 0.025 cm² and 400 cm², more preferentially between 1 and 200 cm², better still between 2 and 50 cm² and even better still between 4 and 25 cm², so as to have optimized compacting of the composition. It has been observed that when the area of the article is within the above ranges, the compacting of the anhydrous solid composition made of powder is lower and the transformation of the powder into a fluid composition in the hands is easier, without any formation of agglomerates.

Preferably, the height of the envelope is greater than or equal to 2 mm, more preferentially ranging from 2 to 10 mm and better still from 3 to 7 mm.

Preferably, the film(s) used in the context of the present invention are chosen from synthetic films such as PVA or PVOH films, and also mixtures thereof.

Preferably, the envelope consists of several layers, for example two or three layers, of films which are each preferably made of different materials.

Advantageously, at least one of these films is a film comprising or consisting of PVA and/or PVOH.

Preferably, the film(s) are sealed so as to form one or more cavities which will comprise the anhydrous solid composition of the invention and will prevent it from escaping.

Advantageously, the packaging article comprises from 1 to 5 g and preferably from 2 to 4.5 g of anhydrous solid composition; and from 0.1 to 0.8 g and preferably from 0.2 to 0.5 g of envelope.

The present invention also relates to a cosmetic process for treating keratin fibres, in particular human keratin fibres such as the hair, comprising a step of using a packaging article as defined above; preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to stand,
iv) rinsing said keratin fibres, and
v) optionally drying said keratin fibres.

It is understood that the composition that is suitable for dissolving the envelope depends on the nature of the envelope. In other words, the composition that is suitable for dissolving the envelope is water or an aqueous composition when the packaging article predominantly or solely contains a hydrophilic envelope. Further, the composition that is suitable for dissolving the envelope is an anhydrous organic composition or an aqueous composition comprising at least one liquid fatty substance or at least one organic solvent other than liquid fatty substances such as lower monoalcohols, for example ethanol, or such as polyols, for example propylene glycol or glycerol, when the packaging article predominantly or solely contains a lipophilic envelope.

Thus, the aqueous composition may simply be water. The aqueous composition may optionally comprise at least one polar solvent. Among the polar solvents that may be used in this composition, mention may be made of organic compounds that are liquid at room temperature (25° C.) and at least partially water-miscible.

Examples that may be mentioned more particularly include alkanols such as ethyl alcohol and isopropyl alcohol, aromatic alcohols such as benzyl alcohol and phenylethyl alcohol, or polyols or polyol ethers, for instance ethylene glycol monomethyl ether, monoethyl ether and monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

More particularly, if one or more solvents are present, their respective content in the aqueous composition ranges from 0.5% to 20% by weight and preferably from 2% to 10% by weight relative to the weight of said aqueous composition.

The dilution ratio (expressed by weight) between one or more packaging articles, as defined previously, and the composition that is suitable for dissolving the packaging article(s) is preferably between 10/90 and 90/10 and more preferentially between 10/90 and 50/50. Better still, this dilution ratio is 20/80.

In particular, the composition obtained on conclusion of the mixing (step i) of the process) may be applied to wet or dry keratin fibres. It is advantageously left in place on the keratin fibres for a time generally ranging from 1 to 15 minutes, preferably from 2 to 10 minutes.

The keratin fibres are then rinsed with water. They may optionally be washed with a shampoo, followed by rinsing with water, before being dried or left to dry.

A subject of the present invention is also the use of a packaging article, as defined previously, for washing and/or conditioning keratin fibres, and in particular human keratin fibres such as the hair.

The examples that follow serve to illustrate the invention without, however, being limiting in nature.

### EXAMPLES

### Example 1

The following anhydrous solid compositions A1 and A2 according to the invention were prepared from ingredients whose contents are indicated in the table below (% in g of active material).

TABLE 1

| | Composition | Composition |
|---|---|---|
| | A1 | A2 |
| Sodium cocoyl | 10.2 | 7.3 |
| isethionate | | |
| Sodium lauroyl | 23 | 16.5 |
| glutamate | | |
| Cocamidopropyl betaine | 10.1 | 7.2 |
| Hydroxypropyl guar | 0.8 | 0.8 |
| hydroxypropyltrimonium | | |
| chloride | | |
| Propylene glycol | — | 3.0 |
| Cetearyl glucoside | — | 1.2 |
| Corn starch | qs 100 | qs 100 |
| R | 2.25 | 2.26 |

Compositions A1 and A2 thus obtained are in anhydrous powder form (their water content (originating from the starting materials) is less than 0.2% by weight, relative to the total weight of the composition) and they may be used for washing hair.

After mixing with water, in the hand, for example, they break down easily and rapidly, producing an abundant, creamy foam, which may then be applied to the hair, where it is easily spread. After rinsing, the hair thus washed is smooth and supple and readily undergoes disentangling.

Compositions A1 and A2 are then packaged in powder form in a water-soluble PVOH-based sachet. The packaging article thus obtained can then be used as a washing composition: it is placed in the palm of the hand, water is added to dissolve it and optionally to form a foam, and it is then applied onto the hair, which has preferably been moistened beforehand.

### Example 2

Composition (A2) according to the invention and comparative compositions (B1) to (B2) were prepared from ingredients whose contents are indicated in the table 2 below (in g of active material)

TABLE 2

| | Composition | Composition | Composition |
|---|---|---|---|
| | A2 | B1 | B2 |
| | (invention) | (comparative) | (comparative) |
| Sodium cocoyl | 7.3 | 7.3 | 7.3 |
| isethionate | | | |
| Sodium lauroyl | 16.5 | 16.5 | 16.5 |
| glutamate | | | |
| Cocamidopropyl | 7.2 | 7.2 | 7.2 |
| betaine | | | |
| Hydroxypropyl guar | 0.8 | 0.8 | 0.8 |
| hydroxypropyltrimonium | | | |
| chloride | | | |
| Propylene glycol | 3.0 | 3.0 | 3.0 |
| Cetearyl glucoside | 1.2 | 1.2 | 1.2 |
| Corn starch | qs 100 | — | — |
| Mica | — | qs 100 | — |
| Kaolin | — | — | qs 100 |
| R | 2.26 | 2.26 | 2.26 |

b) Protocol

0.2 g of each of compositions (A2) and (B1)-(B2) were applied to tresses of natural hair weighing 2.7 g which had been wetted beforehand. The tresses were subsequently massaged with the fingers for 20 seconds before being rinsed with water and dried.

The feel of the hair tresses (wet and dry) was then evaluated by a panel of three experts, and compared with the feel obtained with a conventional shampoo (DOP shampoo, pairwise comparison).

For each composition, each expert assigned a score ranging from −3 to 3 depending on the quality of the feel; 0 corresponds to a feel equivalent to that obtained with the conventional shampoo, while −3 and 3 correspond to a feel respectively less good or better than that obtained with the conventional shampoo.

c) Results

The results obtained are given in the table below (average value of the scores assigned by the 3 experts).

TABLE 3

| | Feel on | Feel on |
|---|---|---|
| | wet hair | dry hair |
| Composition A2 | +2 | +1 |
| Composition B1 | 0 | +1 |
| Composition B2 | +1 | −1 |

The results obtained above show that the composition according to the invention (A2), comprising notably a particular organic filler, imparts a more pleasant feel to the hair than the comparative compositions (B1) and (B2), which comprise mineral fillers.

### Example 3

The anhydrous solid composition A3 according to the invention, below, was prepared from ingredients whose contents are indicated in the table below (% in g of active material).

TABLE 4

| | Composition A3 |
|---|---|
| Sodium cocoyl | 10.2 |
| isethionate | |
| Sodium lauroyl | 23 |
| glutamate | |
| Cocamidopropyl betaine | 10 |
| Hydroxypropyl guar | 0.6 |
| hydroxypropyltrimonium | |
| chloride | |
| Magnesium stearate | 5 |
| Sodium bicarbonate | 3.75 |
| Citric acid | 1.25 |
| Sodium chloride | 1.8 |
| Fragrance | 3.5 |
| Corn starch | qs 100 |
| R | 2.25 |

3 g of composition A3 thus obtained are mixed with water. The composition breaks down easily and rapidly in the hands, producing an abundant, creamy foam. The foam is then applied to the hair, and is easily spread thereon. After rinsing, the hair thus washed is smooth and supple and readily undergoes disentangling.

### Example 4

a) Compositions

Composition (A4) according to the invention and comparative composition (B3) were prepared from ingredients whose contents are indicated in the table 5 below (% in g of active material).

TABLE 5

| | Composition A4 | Composition B3 |
|---|---|---|
| | (invention) | (comparative) |
| Sodium cocoyl | 8.4 | 10.2 |
| isethionate | | |
| Sodium lauroyl | 6.9 | 5.1 |
| glutamate | | |
| Cocamidopropyl betaine | 10.1 | 10.1 |
| Hydroxypropyl guar | 0.8 | 0.8 |
| hydroxypropyltrimonium | | |
| chloride | | |
| Corn starch | qs 100 | qs 100 |
| Amount of anionic | 15.3 | 15.3 |
| surfactants | | |
| R | 0.8 | 0.5 |

b) Evaluation of the Foam Quality

Each of compositions (A4) and (B3) thus obtained were applied to wet hands, before being applied on ½ malleable heads whose hair had been wetted beforehand, at a rate of 2 g of composition per ½ head.

The ½ heads were then massaged 12 seconds with the fingers to generate foam.

The foam quality has then been evaluated visually. The foam obtained with the composition A4 according to the present invention is more abundant, creamier and presents a better hold than the foam obtained with the comparative composition B3.

c) Evaluation of the Cosmetic Properties

0.2 g of each of compositions (A2) and (B3) thus obtained were applied on wet hands, before being applied on medium sensitized locks of hair (alkaline solubility=20%, SA 20), which had been wetted beforehand.

The locks were subsequently massaged with the fingers for 15 seconds. After a leave-on time of 5 minutes, the locks were rinsed with water at 35° C. for 10 seconds (corresponding to 15 passes between the fingers) and then wrung out.

The level of lightness and suppleness conferred to the hair have then been evaluated on wet hair by three experts, in a blind test manner. Each of the experts compared the locks of hair and assigned a score to the softer and smoother lock, assigning a +1 for the lock presenting the best performance level, and +2 when the difference was significant.

The detangling has then been evaluated by one expert, who assigned a +1 for the lock presenting the best performance level, and +2 when the difference was significant.

The assessment of smoothness is tactile. The expert grasps a lock of hair between the thumb and forefinger and slides his fingers along the lock from the top to the tips.

The assessment of suppleness (or flexibility) is also tactile. The expert takes the lock of hair in his hands and tries to bend it. He evaluates if the hair is easy to bend, if it is malleable.

To evaluate detangling, the expert slides a comb through the lock of hair from the root to the tips.

The results obtained are given in the table below (average value of the scores assigned by the 3 experts for the smoothness and the suppleness).

| | smoothness | suppleness | detangling |
|---|---|---|---|
| Composition A4 | +2 | +2 | +1 |
| invention | | | |
| Composition B3 | — | — | — |
| comparative | | | |

The results thus obtained show that composition A4 according to the present invention confers better cosmetic properties, notably in terms of smoothness, suppleness and detangling, to the hair than the comparative composition B3.

Thus, composition A4 is not only able to generate a foam presenting better qualities, that is indeed more abundant, firm and creamy, but also confers better cosmetic properties to the hair, compared to the comparative composition B3.

1-17. (canceled)

18. A solid composition comprising:
(i) at least one anionic surfactant of sulfonate type,
(ii) at least one anionic surfactant of carboxylate type,
(iii) at least one amphoteric or zwitterionic surfactant, and
(iv) at least one polymeric organic filler,
wherein the weight ratio of the total amount of anionic surfactant(s) of carboxylate type to the total amount of anionic surfactant(s) of sulfonate type is greater than or equal to 3:5, and
wherein the composition comprises water in a total amount of less than 5% by weight, relative to the total weight of the composition.

19. The composition of claim 18, wherein the at least one anionic surfactant of sulfonate type is chosen from alkylsulfonates, alkylamidesulfonates, alkylarylsulfonates, alpha-olefinsulfonates, paraffinsulfonates, alkylsulfosuccinates, alkylethersulfosuccinates, alkylamidesulfosuccinates, alkylsulfoacetates, sulfolaurates, N-acyltaurates, acylisethionates, salts thereof, or mixtures of two or more thereof.

20. The composition of claim 18, wherein the at least one anionic surfactant of sulfonate type is chosen from polyoxyalkylenated compounds.

21. The composition of claim 18, wherein the at least one anionic surfactant of sulfonate type is chosen from compounds of formula (I):
R₁—COX—R₂—SO₃M (I)
wherein in formula (I):
R₁ represents a linear or branched alkyl group comprising from 8 to 30 carbon atoms,
X represents an oxygen atom, a —N(CH₃)—, or a —NH— group,
R₂ represents a linear or branched alkyl group comprising from 1 to 4 carbon atoms, and
M represents a hydrogen atom, an ammonium ion, an ion derived from an alkali metal or alkaline-earth metal, or an ion derived from an organic amine.

22. The composition of claim 18, wherein the total amount of the anionic surfactant(s) of sulfonate type ranges from 1% to 30% by weight, relative to the total weight of the composition.

23. The composition of claim 18, wherein the at least one anionic surfactant of carboxylate type is chosen from compounds of formula (II):
R—(OCH₂CH₂)ₙW—(CHY₁)ₚ—COOX (II)
wherein in formula (II):
Y₁ represents a hydrogen atom, a group (CH₂)_{q}COOX, or a hydroxyl group;
W represents an oxygen atom, a group (O-Glu-O)ᵣ—(COCH(Y₂)—(C(OH)COOX)ₜ)ₛ, or a group CO—NR₃;
Y₂ represents a hydrogen atom or a hydroxyl group;
R₃ represents a hydrogen atom or a methyl group;
X represents a hydrogen atom, an ammonium ion, an ion derived from an alkali metal or alkaline-earth metal, or an ion derived from an organic amine;
R represents a linear or branched alkyl group comprising from 8 to 30 carbon atoms;
Glu represents a divalent radical derived from glucopyranose by removal of 2 hydroxyl groups;
p is equal to 0 or 1;
q represents an integer ranging from 1 to 10;
n represents an integer ranging from 0 to 50;
r denotes a number ranging from 1 to 10;
s is equal to 0 or 1; and
t is equal to 0 or 1.

24. The composition of claim 18, wherein the at least one anionic surfactant of carboxylate type is chosen from N-acylglycinates, N-acylsarcosinates, or N-acylglutamates.

25. The composition of claim 18, wherein the total amount of anionic surfactant(s) of carboxylate type ranges from 1% to 40% by weight, relative to the total weight of the composition.

26. The composition of claim 18, wherein the total amount of anionic surfactant(s) of carboxylate type ranges from 5% to 30% by weight, relative to the total weight of the composition.

27. The composition of claim 18, wherein the weight ratio of the total amount of anionic surfactant(s) of carboxylate type to the total amount of anionic surfactant(s) of sulfonate type ranges from 3:5 to 5:1.

28. The composition of claim 18, wherein the at least one amphoteric or zwitterionic surfactant is chosen from alkyl(C₈-C₂₀)betaines, alkyl(C₈-C₂₀)amidoalkyl(C₃-C₈)betaines, or mixtures of two or more thereof.

29. The composition of claim 18, wherein the total amount of surfactants is up to 60% by weight, relative to the total weight of the composition.

30. The composition of claim 18, wherein the total amount of surfactants ranges from 30% to 50% by weight, relative to the total weight of the composition.

31. The composition of claim 18, wherein the at least one polymeric organic filler is chosen from cyclodextrins, starches, alginates, gellans, guar gums, celluloses, wood flours, crosslinked polyvinylpyrrolidones, polyacrylates, or mixtures of two or more thereof.

32. The composition of claim 18, wherein the total amount of polymeric organic filler(s) is at least 20% by weight, relative to the total weight of the composition.

33. The composition of claim 18, wherein the total amount of polymeric organic filler(s) ranges from 30% to 70% by weight, relative to the total weight of the composition.

34. The composition of claim 18, further comprising at least one cationic polymer other than the polymeric organic filler(s).

35. A cosmetic method for treating keratin fibers, comprising applying to the keratin fibers a solid composition comprising:
(i) at least one anionic surfactant of sulfonate type,
(ii) at least one anionic surfactant of carboxylate type,
(iii) at least one amphoteric or zwitterionic surfactant, and
(iv) at least one polymeric organic filler,
wherein the weight ratio of the total amount of anionic surfactant(s) of carboxylate type to the total amount of anionic surfactant(s) of sulfonate type is greater than or equal to 3:5,
wherein the composition further comprises water in a total amount of less than 5% by weight, relative to the total weight of the composition, and
wherein the solid composition is moistened with water, then applied to the keratin fibers.

36. A packaging article comprising:
at least one cavity,
a solid composition (A) comprising:
(i) at least one anionic surfactant of sulfonate type,
(ii) at least one anionic surfactant of carboxylate type,
(iii) at least one amphoteric or zwitterionic surfactant, and
(iv) at least one polymeric organic filler,
wherein the weight ratio of the total amount of anionic surfactant(s) of carboxylate type to the total amount of anionic surfactant(s) of sulfonate type is greater than or equal to 3:5,
wherein the composition further comprises water in a total amount of less than 5% by weight, relative to the total weight of the composition,
wherein the solid composition (A) is disposed in one of the cavities of the packaging article, and
wherein a material of the packaging article comprises one or more water-soluble and/or liposoluble compounds.

37. A cosmetic method for treating keratin fibers comprising:
i) mixing the packaging article according to claim 36 in a composition capable of dissolving the packaging article,
ii) applying the composition obtained in step i) to the keratin fibers,
iii) rinsing the keratin fibers, and
iv) optionally drying the keratin fibers.
